(19) United States
(12) Patent Application Publication
(10) Pub. No.: US 2006/0276844 A1
(43) Pub. Date: Dec. 7, 2006

(54) Ingestible device for nitric oxide production in tissue

(76) Inventors: Ruth Alon, Michmoret (IL); Rina Lev, Haifa (IL); Ziv Belsky, Haifa (IL)

Correspondence Address: RATNERPRESTIA, P O BOX 980, VALLEY FORGE, PA, 19482-0980, US

(21) Appl. No.: 11/436,439

(22) Filed: May 18, 2006

Related U.S. Application Data
(60) Provisional application No. 60/682,421, filed on May 19, 2005.

(51) Int. Cl. A61N 1/00

(52) U.S. Cl. 607/2

(57) ABSTRACT

Apparatus is provided including an ingestible device, which includes two or more electrodes, and a signal controller, configured to drive the electrodes to apply an electrical signal to an inner surface of a wall of a gastrointestinal (GI) tract of a subject, and to configure the signal to induce local endogenous release of nitric oxide (NO). Other embodiments are also described.

### CROSS-REFERENCES TO RELATED APPLICATIONS

The present patent application claims the benefit of U.S. Provisional Application 60/682,421, filed May 19, 2005, entitled, “Ingestible electro-stimulator for acute or chronic therapies through enhancement or triggering of biological processes in surrounding tissue,” which is assigned to the assignee of the present application and is incorporated herein by reference.

The present application is related to an international patent application filed on even date herewith, entitled, “Ingestible device for nitric oxide production in tissue,” which is assigned to the assignee of the present application and is incorporated herein by reference.

### FIELD OF THE INVENTION

The present invention relates generally to techniques for stimulating the gastrointestinal (GI) tract, and specifically to an ingestible device for stimulating the GI tract.

### BACKGROUND OF THE INVENTION

Nitric oxide (NO) is an important mediator of several physiological processes in the gastrointestinal (GI) tract (Konturek et al., 1995; complete citations of all articles are provided hereinbelow). Endogenous NO is derived from enzymatic conversion of L-arginine to L-citrulline by NO synthase (NOS), a family of isoenzymes. Recent immunohistochemistry and immunofluorescence studies have shown that two constitutively expressed, Ca2+ dependent NOS isoforms—neuronal NOS (nNOS, NOS1) and endothelial NOS (eNOS, NOS3)—are widely expressed in epithelial cells of lamina propria of the intestinal villi, and myenteric and submucosal neurons (Chen et al., 2002; Qu et al., 1999). These isoforms are involved in regulation of vascular perfusion, bowel motility, and fluid and electrolyte transport. The third Ca2+ independent, inducible NOS isoform (iNOS, NOS2) is present in macrophages, mast cells, endothelial, and epithelial cells. The induction of iNOS generally occurs in states of intestinal inflammation, hyperpermeability, immune activation, and tissue injury (Beckett et al., 1998; Ding et al., 2005).

nNOS and eNOS isoforms have been shown to be critical to normal physiology of the gastrointestinal tract. Inhibition of these enzymes may cause tissue damage and inflammation (Kubes et al., 2000; Leffer et al., 1999). Using a transgenic mice animal model, Beck P L et al. (2004) demonstrated that the loss of nNOS resulted in more severe inflammatory diseases of the intestine and increased mortality, whereas the loss of eNOS or iNOS was protective. Additional studies have shown that nNOS plays an essential role in regulation of bowel motility and sphincter function (Mashimo et al., 1999; Mearin et al., 1993).

A number of studies have demonstrated that NO is involved in intestinal water transport (Mourad, 1999). NO can act both as a secretagogue and an absorbagogue depending on concentration, local circumstances, and on the site of delivery (Turvill et al., 1999; Dijkstra et al., 2004; Vilijoen et al., 2001; Schirgi-Degen et al., 1998).

NO plays a key role in the modulation of mucosal blood flow, either in basal conditions or after challenge with irritants. Blockade of NO synthesis significantly decreases blood flow in the gastric mucosa, the mesenteric vascular bed, and several areas of intestinal tissue. NO is responsible for endothelium derived tonic relaxation of all types of blood vessels by stimulating and increasing cGMP in smooth muscle cells (Barrachina et al., 2001).

Cerwinka et al. (2002) showed that eNOS-derived NO plays a modulatory role in endotoxin-induced platelet-endothelial cell adhesion in intestinal venules, and that the activation of the soluble guanylate cyclase (sGC) pathway is responsible for the antiadhesive action of NO.

NO donors (i.e., NO releasing substances) have been developed for various practical applications in biology and drug design (Wang et al., 2005).

In-vitro studies showed that the addition of NO donors (sodium nitroprusside (SNP), S-nitroso-acetyl-penicillamine (SNAP), molsidomine (SIN)), or saturated NO solutions to mouse ileum results in a decrease in transepithelial electrical resistance, implying that NO has a proabsorptive effect (Unno et al., 1997).

Additional studies have demonstrated that NO donors (NOC5, NOC7, NOC12) can improve absorption of macromolecules from all regions of the rat intestine. The degree of absorption-enhancing effect of NO donors is dependent on the molecular weights of compounds. Furthermore, studies have shown that the absorption-enhancing mechanism of NO donors includes the dilation of the tight junction in the epithelium via a paracellular route. The effect of NO donors was found to be reversible and nontoxic to the intestinal mucosa (Yamamoto et al., 2001; Numata et al., 2000; Takahashi et al., 2004).

The most studied NO donor, glyceryl trinitrate, which has been used for many years as a vasodilatator, has been found to be effective in acceleration of the healing of pre-existing ulcers in the gastrointestinal tract (Elliott et al., 1995) and anal fissures (Lund et al., 1997). The coupling of NO donors to nonsteroidal anti-inflammatory drugs (NSAIDs) has proven to be useful for reducing the gastrointestinal toxicity of these drugs without decreasing their efficacy (Muscara et al., 1999). Gookin et al. (2002) have shown that NO is a key mediator of early villous reepithelialization following acute mucosal injury in porcine ileum.

An immunomodulatory protective role for NO has been shown by various in vivo studies, in which NO has been identified as an important mast cell mediator related to gastrointestinal mucosal protection and the mucosal immune system (Wallace, 1996).

Recent in vitro and in vivo studies have shown the existence of extensive and complex non-adrenergic non-cholinergic (NANC) innervation of the muscular layer in the various parts of the gastrointestinal tract. Electrical field stimulation applied to the NANC nerves of the smooth muscles leads to release of NO (Takahashi, 2003).

The release of NO in intestinal tissue has been studied in different functional experiments, in which low frequency (10-30 Hz) electrical stimulation at very high currents (100-200 mA) was applied on myenteric plexus-longitudinal muscle preparations of rodent ileum and colon. Intermittent field stimulation at 10 Hz, 1 ms, for 30 minutes led to a significant increase in NO content in the muscle-myenteric plexus strips. Electrically-induced NO synthesis and release was nearly prevented by the NO synthase inhibitor NG-nitro-L-arginine (L-NNA). Moreover, electrically-induced NO formation was largely inhibited by removal of extracellular calcium, implying that the neuronal Ca-dependent NO synthase (NNOS) was involved (Hallen et al., 2001; Hebeiss et al., 1999; Olgart et al., 1998).

NO-producing electrical stimuli have been generated by external stimulators and delivered to electrodes implanted at seromuscular or subserosal layers of the gastrointestinal tract (Liu et al., 2005; Xing et al., 2006).

The electrically-evoked release of NO may have either a relaxatory effect (Sanders et al., 1992; Liu et al., 2005) or a contraction-inducing effect (Ekblad et al., 1997; Zhang et al., 2001) on the gastrointestinal muscles, with consequent modulation of peristaltic waves. Additionally, electrical field stimulation (EFS)—induced NO plays an important role in regulating contraction and relaxation of the GI sphincters (Mizhorkova et al., 1994; Ishiguchi et al., 2000; Tomita et al., 1999; Tanobe et al., 1995; Rattan et al., 2004; Nakamura et al., 1998).

Ingestible electronic pills have been developed as diagnostic measuring systems for real time analysis of temperature, pH, conductivity, and intraluminal pressure (Rav-Acha et al., 2003; Andres and Bingham, 1970; Johannessen et al., 2002; Wang et al., 2003; Arshak et al., 2005; Nair et al., 2002), and imaging of different regions of the GI (tract (Swain, 2003; Kimchy et al., 2002; Zilberstein et al., 2005).

Ingestible autonomous electrical stimulators have been designed for normalizing motility, secretory and metabolic function of the gastrointestinal tract (PCT Publication WO 97/27900 to Karev; Gluschnik et al., 2003; Zherlov et al., 2005; U.S. Pat. No. 6,453,199 to Kobozev).

An increase in the amount of a substrate for NO or in the enzymatic activity of NO synthase can lead to an increase in the formation of endogenous NO in various systems throughout the body.

Fabio et al. (2004) demonstrated that oral administration of L-arginine (the substrate for the synthesis of NO) to humans is associated with an increased concentration of NO in exhaled air and with an increase in the concentration of L-arginine and nitrate in plasma. Such administration of L-Arginine provides sufficient substrate for NO synthase enzymes to produce NO, which in turn has therapeutic and/or beneficial effects on various systems throughout the body.

The effect of L-arginine therapy on endothelial function has been studied in healthy and diseased states.

Marchesi et al. (2001) demonstrated that transient impairment of endothelial function, associated with an early stage of atherosclerosis, is partly abolished by oral L-arginine administration. Kawano et al. (2002) showed that L-arginine improves endothelial function in hypercholesterolemic subjects.

Aging is associated with progressive endothelial dysfunction in normal humans. Endothelial cell function was improved by oral L-arginine supplementation in a group of healthy elderly subjects (Bode-Boger et al., 2003).

Adams et al. (1997) reported substantial improvements in endothelium-dependent vasodilation and reduced monocyte/endothelial cell adhesion after L-arginine supplementation in young men with coronary artery disease. Mild to moderate elevations of plasma homocysteine levels in healthy subjects activates coagulation, and modifies the adhesive properties of endothelium.

West et al. (2005) demonstrated that oral L-arginine administration is associated with a significant reduction in plasma homocysteine and a moderate reduction in diastolic blood pressure, as well as a decrease in platelet aggregation and monocyte adhesiveness. Moreover, as shown by Huynh et al. (2002), oral arginine may increase endothelial NO synthase (NOS) activity to increase vascular NO and temporally reduce blood pressure in mildly hypertensive type 2 diabetic patients.

Piatti et al. (2002) reported that long-term oral L-Arginine treatment significantly improves peripheral and hepatic insulin sensitivity in type 2 diabetic patients.

Studies in humans have shown that oral L-arginine supplements improve vascular health through NO generation. Clinical trials to date support potential clinical applications of L-arginine in the treatment of coronary artery disease and peripheral arterial disease, as well as in the prevention of in-stent restenosis.

U.S. Pat. No. 6,340,480 to Duckett et al., which is incorporated herein by reference, describes an orally administrated composition including L-arginine, ginseng and Zizyphi fructus, and suggests that the composition promotes systemic vascular relaxation and dilation, and is effective in reduction of hypertension.

Bing et al. (2000) have shown that the orally administered NO donor B-NOD, in combination with an Aspirin moiety, can be useful in the long-term treatment of coronary artery disease and in clinical situations in which long term release of NO may be beneficial.

As reported by Reitz et al. (2005), NO-based processes, including the NO/cGMP pathway, have been implicated in the genitourinary system. Oral intake of Sildenafil, which is a phosphodiesterase inhibitor involved in the NO/cGMP pathway, resulted in a relevant increase in periurethral blood flow, as determined using color Doppler measurements. After oral administration in healthy humans, an NO donor had a functionally relevant effect on the resting tone and contractile behavior of the human external urethral sphincter in vivo. In another functional study in humans with spinal cord injury, subvesical obstruction caused by detrusor-sphincter dyssynergia was successfully reduced by oral administration of a NO donor.

Additionally, Chen et al. (1999) reported that oral administration of L-arginine in high doses may cause significant subjective improvement in sexual function in men with organic erectile dysfunction associated with decreased NO production.

According to Jablechka et al. (2004), long-term oral supplementation of L-arginine leads to substantial increases in NO concentrations and total antioxidant status levels in the blood of patients with atherosclerotic peripheral arterial disease.

US Patent Application Publication 2004/0267240 to Gross et al., which is assigned to the assignee of the present patent application and is incorporated herein by reference, describes apparatus for drug administration, including an ingestible capsule, which includes a drug, stored by the capsule, and an environmentally-sensitive mechanism, adapted to change a state thereof responsively to a disposition of the capsule within a gastrointestinal (GI) tract of a subject. The capsule further includes first and second electrodes, and a control component, adapted to facilitate passage of the drug, in response to a change of state of the environmentally-sensitive mechanism, through an epithelial layer of the GI tract by driving the first and second electrodes to apply a “low intensity time-varying” (LITV) signal.

US Patent Application Publication 2005/0058701 to Gross et al., which is assigned to the assignee of the present patent application and is incorporated herein by reference, describes apparatus for drug administration, including an ingestible capsule, which includes a drug, stored by the capsule, and an environmentally-sensitive mechanism, adapted to change a state thereof responsively to a disposition of the capsule within a gastrointestinal (GI) tract of a subject. The capsule further includes first and second electrodes, and a control component, adapted to facilitate passage of the drug, in response to a change of state of the environmentally-sensitive mechanism, through an epithelial layer of the GI tract by driving the first and second electrodes to apply a series of pulses at a current of less than about 5 mA, at a frequency of between about 12 Hz and about 24 Hz, and with a pulse duration of between about 0.5 milliseconds and about 3 milliseconds.

U.S. Pat. No. 6,865,416 to Dev et al., which is incorporated herein by reference, describes methods for inducing or increasing the vasodilation of a vessel, such as a blood vessel or a gastrointestinal vessel. Also described are methods for inducing or increasing the flow of fluid through a vessel. An electrical impulse is applied to the vessel in order to induce or increase vessel vasodilation or to induce or increase the flow of fluid through the vessel. In one embodiment, a double-balloon catheter system incorporating electroporation technology is used to apply the electrical impulse endoluminally.

PCT Publication WO 05/112895 to Zilberstein et al., which is incorporated herein by reference, describes an ingestible pill platform for colon imaging, designed to recognize its entry to the colon and expand in the colon, for improved imaging of the colon walls. On approaching the external anal sphincter muscle, the ingestible pill may contract or deform, for elimination. Colon recognition may be based on a structural image, based on the differences in diameters between the small intestine and the colon, and particularly, based on the semilunar fold structure, which is unique to the colon.

US Patent Application Publication 2004/0186530 and PCT Publication WO 03/015861 to Gluschuk et al., which are incorporated herein by reference, describe an electrostimulating device comprising a casing and at least two stimulating electrodes. At least one of the stimulating electrodes is mobile and external to the casing. The mobile electrode is tethered to the device with an insulated conducting cable and is operative to increase the distance between the stimulating electrodes, so as to stimulate a greater volume of cells.

PCT Publication WO 97/27900 to Karev, which is incorporated herein by reference, describes an electronic “normalizer” for use in the treatment of the gastro-intestinal tract, in gynecology for stimulating the bioelectrical, motor and secretory activity of organs, for cleansing duct systems, stimulating the pancreas and prostate gland, modifying psycho-physiological and immune state, or prevention and treatment of malignancies. The electronic normalizer comprises a housing, two electrodes, an insert, a microprocessor, a contact element, power source, and a spring.

PCT Publication WO 02/058531 to Kimchy et al., which is incorporated herein by reference, describes an ingestible device, adapted to travel in the gastrointestinal tract and perform a diagnostic image of tissue therein. The diagnostic image may comprise diagnostic information as a function of time, or diagnostic information as a function of distance traveled within the gastrointestinal tract.

U.S. Pat. No. 6,453,199 to Kobozev, which is incorporated herein by reference, describes an electrical stimulation capsule comprising a casing with electrodes, the casing containing a power source, a control unit of which M outputs are connected to M electrodes, a device for receiving signals from internal organs and/or an external transmitter, to (1−N) outputs of which are connected (1−N) inputs of the control unit. In an embodiment, the capsule contains P additional electrodes provided with a coating of microelements or medicinal preparations and connected to P separate outputs of the control unit.

PCT Publication WO 02/07598 to Nair et al., which is incorporated herein by reference, describes an ingestible capsule, and a method for determining medical information from within the alimentary canal utilizing the ingestible capsule. The capsule includes a non-digestible outer shell that is configured to pass through the alimentary canal. A marker membrane is exposed through a portion of the non-digestible outer shell. The marker membrane is characterized as detecting and identifying predetermined detectable information. Housed within the non-digestible outer shell are a bio-sensor that alters its electronic properties in the presence of specific information obtained by the marker membrane from within the alimentary canal, a low frequency transducer that sends a signal of the changed electronic properties to a receiver positioned outside the body, and a miniature battery for powering the transducer.

Ingestible capsules containing a transmitter and other electrical components have been described. In 1964 researchers at Heidelberg University developed a pill for monitoring pH of the GI tract (Noller, H. G., “The Heidelberg Capsule Used For the Diagnosis of Peptic Diseases,” Aerospace Medicine, February 1964, pp. 115-117). U.S. Pat. No. 4,844,076 to Lesho et al., which is incorporated herein by reference, describes a temperature responsive transmitter, encapsulated in an ingestible size capsule.

The use of electrostimulating capsules for promoting peristalsis has been described. PCT Publications WO 97/31679 to Dirin and WO 97/26042 to Terekhin, both of which are incorporated herein by reference, describe ingestible capsules for electrostimulation of the alimentary tract, to be used, for example, as a post-surgical therapy, as a prophylactic measure of alimentary tract diseases, or for the promotion of peristalsis.

PCT Publication 01/08548 to Mosse et al., which is incorporated herein by reference, describes a self-propelling device that is adapted to travel through a passage having walls containing contractile tissue. The device comprises a body and at least one contractile-tissue stimulating means for stimulating the walls to urge the device in a forward direction. The stimulating device may comprise electrodes, and the passage may be the gut.

PCT Publication WO 97/31679 further discloses that USSR Inventor's Certificate No. 1223922, Int. Cl. A 61 N 1/36, Bulletin No. 14, by Pekarasky et al., entitled, “Gastrointestinal tract Electrostimulator,” which is incorporated herein by reference, describes a swallowable capsule adapted for electrostimulation of the alimentary tract, as post-surgical therapy, as a prophylactic measure of alimentary tract diseases, or for the promotion of peristalsis, which is further adapted for the dispensing of medication.

PCT Publication WO 02/098501 to Keisari et al., which is incorporated herein by reference, describes a method for treating tumor tissue, including applying to cells of the tumor tissue electrical field pulses having a strength, a repetition frequency, and a pulse width selected to be capable of inducing endocytosis-mediated cell death, thereby treating the tumor tissue.

The following articles, all of which are incorporated herein by reference, may be of interest:
Adams et al., “Oral L-arginine improves endothelium-dependent dilatation and reduces monocyte adhesion to endothelial cells in young men with coronary artery disease,” Atherosclerosis 129(2):261-9 (1997)
Andres M R et al., “Tubeless gastric analysis with a radiotelemetering pill (Heidelberg capsule),” CMAJ 102:1087-1089 (1970)
Arshak A et al., “Review of the potential of a wireless MEMS and TFT microsystems for the measurement of pressure in the GI tract,” Medical Engineering & Physics 27(5):347-356 (2005)
Barrachina M D et al., “Role of nitric oxide in gastrointestinal inflammatory and ulcerative diseases: perspective for drugs development,” Current Pharmaceutical Design 7:31-48 (2001)
Beck P L et al., “Paradoxical roles of different nitric oxide synthase isoforms in colonic injury,” Am J Physiol (Gastrointest. Liver Physiol.) 286:G137-G147 (2004)
Beckett C G et al., “The detection and localization of inducible nitric oxide synthase production in the small intestine of patients with coeliac disease,” Eur J Gastroenterol Hepatol 10(8):641-7 (1998)
Bing et al., “The pharmacology of a new nitric oxide donor: B-NOD,” Biochemical and Biophysical Research Communications 275:350-353 (2000)
Bode-Boger et al., “Oral L-arginine improves endothelial function in healthy individuals older than 70 years,” Vasc Med 8(2):77-81 (2003)
Cerwinka W H et al., “Nitric oxide modulates endotoxin-induced platelet-endothelial cell adhesion in intestinal venules,” Am J Physiol Heart Circ Physiol 282:H1111-H1117 (2002)
Chen J et al., “Effect of oral administration of high-dose nitric oxide donor L-arginine in men with organic erectile dysfunction: results of a double-blind, randomized, placebo-controlled study,” BJU Int 83(3):269-73 (1999)
Chen Y-M et al., “Distribution of constitutive nitric oxide synthase in the jejunum of adult rat,” Worl J Gastroenterol 8(3):537-539 (2002)
Ding X et al., “Inducible nitric oxide synthase-dependent DNA damage in mouse model of inflammatory bowel disease,” Cancer Sci 96(3): 157-63 (2005)
Dijkstra G et al., “Targeting nitric oxide in the gastrointestinal tract. Current Opinion in Investigational Drugs (in press), Dissertation, 2004
Ekblad E et al., “Motor responses in rat ileum evoked by nitric oxide donors vs. field stimulation: modulation by pituitary adenylate cyclase-activating peptide, forskolin and guanylate cyclase inhibitors,” JPET 283:23-28 (1997)
Elliott S N et al., “A nitric oxide-releasing nonsteroidal anti-inflammatory drug accelerates gastric ulcer healing in rats,” Gastroenterology 109(2):524-30 (1995)
Fabio R L et al., “Nitric oxide in health and disease of the respiratory system,” Physiol Rev 84:731-765 (2004)
Gookin J L et al., “Inducible nitric oxide synthase mediates early epithelial repair of porcine ileum,” Am J Physiol Gastrointest Liver Physiol 283:G157-G168 (2002)
Hah J M et al., “Reduced amide bond peptidomimetics. (4S)-N-(4-amino-5-[aminoethyl]aminopentyl)-N′-nitroguanidines, potent and highly selective inhibitors of neuronal nitric oxide synthase,” J Med Chem 44(16):2667-2670 (2001)
Hallen K et al., “Modulation of neuronal nitric oxide release by soluble guanylyl cyclase in guinea pig colon,” Biochem Biophys Res Commun 280(4): 1130-1134 (2001)
Hebeiss K et al., “Cholinergic and GABAergic regulation of nitric oxide synthesis in the guinea pig ileum,” Am J Physiol 276:(Gastrointest Liver Physiol 39):G862-G866 (1999)
Huynh N T et al., “Oral arginine reduces systemic blood pressure in type 2 diabetes: its potential role in nitric oxide generation,” Journal of the American College of Nutrition 21(5):422-427 (2002)
Ishiguchi T et al., “Nitrergic and purinergic regulation of the rat pylorus,” AJP—GI 279:740-747 (2000)
Jablecka A et al., “The influence of two different doses of L-arginine oral supplementation on nitric oxide (NO) concentration and total antioxidant status (TAS) in atherosclerotic patients,” Med Sci Monit 10(1):CR29-32 (2004)
Johannessen E A et al., “An ingestible electronic pill for real time analytical measurements of the gastrointestinal tract,” Proc of the mTAS 2002 Symposium, Japan, 181-183 (2002)
Kawano H et al., “Endothelial dysfunction in hypercholesterolemia is improved by L-arginine administration: possible role of oxidative stress,” Atherosclerosis 161 (2):375-80 (2002)
Konturek S K et al., “Role of nitric oxide in the digestive system,” Digestion 56:1-13 (1995)
Kubes P et al., “Nitric oxide and intestinal inflammation,” Am J Med 109(2):150-158 (2000)
Leffer A M et al., “Nitric Oxide. II. Nitric oxide protects in intestinal inflammation,” Am J Physiol 276 (Gastrointest Liver Physiol 39):G572-G575 (1999)
Liu S et al., “Cross-talk along gastrointestinal tract during electrical stimulation: effects and mechanisms of gastric/colonic stimulation on rectal tone in dogs,” Am J Physiol Gastrointest Liver Physiol 288(6):G1195-8 (2005)
Lund J N et al., “Glyceryl trinitrate is an effective treatment for anal fissure,” Dis Colon Rectum 40(4):468-70 (1997)
Marchesi S et al., “Oral L-arginine administration attenuates postprandial endothelial dysfunction in young healthy males,” J Clin Pharm Ther 26(5):343-9 (2001)
Mashimo H et al., “Lessons from genetically engineered animal models. IV Nitric oxide synthase gene knockout mice,” Am J Physiol (Gastrointest Liver Physiol 40) 277:G745-G750 (1999)
Mearin F et al., “Patients with achalasia lack nitric oxide synthase in the gastroesophageal junction,” Eur J Clin Invest 23:724-728 (1993)
Mizhorkova Z N et al., “Role of nitric oxide in mediating non-adrenergic non-cholinergic relaxation of the cat ileocecal sphincter,” Eur J Pharmacol 265(1-2):77-82 (1994)
Mourad T F, “Role of nitric oxide in intestinal water and electrolyte transport,” Gut 44:143-147 (1999)
Muscara M N et al., “Nitric Oxide V. Therapeutic potential of nitric oxide donors and inhibitors,” Am J Physiol 276 (Gastrointest Liver Physiol 39):G1313-G1316(1999)
Nakamura K et al., “Nicotinic receptor mediates nitric oxide synthase expression in the rat gastric myenteric plexus,” J Clin Invest 101(7):1479-1489 (1998)
Numata N et al., “Improvement of intestinal absorption of macromolecules by Nitric Oxide Donor,” Journal of Pharmaceutical Sciences 89(10):1296-1304 (2000)
Olgart C et al., “Blockage of nitrergic neuroeffector transmission in guinea-pig colon be a selective inhibitor of soluble guanylyl cyclase,” Acta Physiol Scand 162:89-95 (1998)
Piatti P M et al., “Long-term oral L-arginine administration improves peripheral and hepatic insulin sensitivity in type 2 diabetic patients,” Diabetes Care 24(5):875-880 (2001)
Rattan S et al., “Mechanism of internal anal sphincter relaxation by CORM-1, authentic CO, and NANC nerve stimulation,” AJP—GI 287:605-611 (2004)
Rav-Acha M et al., “Core body temperature monitoring using the telemetric pill,” Harefuah 142(3):197-202, 238 (2003)
Reitz A et al., “Targeting bladder outlet obstruction from benign prostatic enlargement via the nitric oxide/cGMP pathway?” BJU International 96:250-253′ (2005)
Qu X W et al., “Type I nitric oxide synthase (NOS) is the predominant NOS in rat small intestine regulation by platelet-activating factor,” Biochem Biophys Acta 1451(1):211-7 (1999)
Sanders K M et al., “Nitric oxide as a mediator of nonadrenergic noncholinergic neurotransmission,” AJP—Gastrointestinal and Liver Physiology 262(3):G379-G392 (1992)
Schirgi-Degen A et al., “Proabsorptive properties of nitric oxide,” Digestion 59:400-403 (1998)
Swain P, “Wireless capsule endoscopy,” Gut 52:48-50 (2003)
Takahashi T, “Pathophysiological significance of neuronal nitric oxide synthase in the gastrointestinal tract,” Journal of Gastroenterology 38(5)421-430 (2003)
Takahashi K et al., “Characterization of the influence of nitric oxide donors on intestinal absorption of macromolecules,” International Journal of Pharmaceutics 286:89-97 (2004)
Tanobe Y et al., “Functional role and histological demonstration of nitric-oxide-mediated inhibitory nerves in dog sphincter of Oddi,” Neurogastroenterol Motil 7(4):219-27 (1995)
Tomita R et al., “The role of nitric oxide (NO) in the human pyloric sphincter,” Hepatogastroenterology 46(29):2999-3003 (1999)
Turvill J L et al., “Proabsorptive and prosecretory roles of nitric oxide in cholera toxin induced secretion,” Gut 44:33-39 (1999)
Unno N et al., “Nitric oxide-induced hypermeability of human intestinal epithelial monolayers is augmented by inhibition of the amiloride-sensitive Na+-H+ antiport: Potential role of peroxynitrous acid,” Surgery 122:485-492 (1997)
Vilijoen M et al., “A nitric oxide and gastrointestinal hyperpermeability,” The Medicine Journal 43:9, 33-37 (2001)
Wallace J L, “Cooperative modulation of gastrointestinal mucosal defense by prostaglandins and nitric oxide,” Clin Invest Med 19(5):346-51 (1996)
Wang L et al., “A networked wireless microsystem for remote gastrointestinal monitoring,” Transducers '03 12th International Conference on Solid-State Sensors, Actuators and Microsystems, Digest of Technical Papers Pt 2 Vol 2, pp. 1184-7 (2003)
Wang P G et al. (editors), Nitric Oxide Donors: For Pharmaceutical and Biological Applications, Wiley (2005)
West S G et al., “Oral L-arginine improves hemodynamic responses to stress and reduces plasma homocysteine in hypercholesterolemic men,” J Nutr 135:212-217 (2005)
Xing J H et al., “Effects and mechanisms of long-pulse gastric electrical stimulation on canine gastric tone and accommodation,” Neurogastroenterology and Motility 18(2):136 (2006)
Yamamoto A et al., “Modulation of intestinal permeability by nitric oxide donors: implications in intestinal delivery of poorly absorbable drugs,” JPET 296:84-90 (2001)
Zhang Y et al., “Nitric oxide contracts longitudinal smooth muscle of opossum oesophagus via excitation-contraction coupling,” J Physiol 536:133-140 (2001)
Zherlov G K et al., “Correction of the motor-evacuation function of the postoperative stomach in the early postoperative period,” Exsp Klin Gastroenterol (4):44-48, 112 (2005)

### SUMMARY OF THE INVENTION

In embodiments of the present invention, an ingestible electrical-stimulation device comprises a signal controller configured to apply an electrical signal intraluminally to an inner surface of a wall of the gastrointestinal (GI) tract. The signal controller configures the signal to induce and/or enhance local endogenous release of nitric oxide (NO) in the GI tract, in order to treat a local or a systemic condition. Typically, the signal is configured to stimulate mucosal and submucosal neuronal complexes, thereby activating neuronal NO synthase (nNOS) and/or submucosal endothelial NO synthase (eNOS).

The electrically-induced local release of NO in the GI tract generally:
maintains gastrointestinal mucosal integrity and prevents acute microvascular injuries induced by endotoxins, ischemic factors, and various irritants;
modulates mucus and alkaline secretion, thereby enhancing the GI tract's viscoelastic protective layer and accelerating healing of preexisting ulcers in the GI wall;
improves blood flow in the gastric mucosa, the mesenteric vascular bed, and various areas of the intestinal tissue, thereby contributing to the maintenance of mucosal integrity;
causes vasodilation of the surrounding GI vasculature, thereby causing increased perfusion of tissue, which has local anti-necrotic and anti-inflammatory effects;
attenuates inflammatory response and improves microvascular reactions occurring in the GI wall during various pathological conditions, such as GI inflammation, sepsis, irritable bowel syndrome (IBS), Crohn's disease, and other inflammatory disorders;
down-regulates the immune response during various inflammatory and immunogenic conditions;
regulates muscle tone of the GI sphincters, and the peristaltic reflex of the stomach and the intestine, including gastric emptying and intestinal transit, thereby treating various motility disorders of the gastrointestinal tract; and/or
has beneficial effects outside of the GI tract, such as:
   systemic anti-inflammatory effects beneficial for treating inflammatory diseases;
   improvement of endothelial function with consequent vasodilatation and increases in blood flow, in various disease states such as hypertension, atherosclerosis, hypercholesterolemia, diabetes, peripheral vascular diseases, coronary artery diseases, and urogenital disorders;
   an inhibitory effect on platelet aggregation and an anticoagulatory effect, which are beneficial for treating a coagulation- anticoagulation imbalance in various pathological states;
   a systemic antioxidative effect by reducing free radical reactions and stimulating antioxidative enzymes, which provides therapeutic benefits for various disorders associated with increased formation of free radicals, such as atherosclerosis, peripheral vascular disorders, and diabetes; and/or
   a beneficial effect on insulin sensitivity in diabetes.

There is therefore provided, in accordance with an embodiment of the present invention, apparatus including an ingestible device, which includes:

two or more electrodes; and

a signal controller, configured to drive the electrodes to apply an electrical signal to an inner surface of a wall of a gastrointestinal (GI) tract of a subject, and to configure the signal to induce local endogenous release of nitric oxide (NO) in the GI tract.

In an embodiment, the signal controller is configured to configure the signal to stimulate neuronal complexes of the GI tract selected from the group consisting of: mucosal neuronal complexes, and submucosal neuronal complexes.

For some applications, the signal controller is configured to drive the electrodes to apply the signal with an amplitude of between 2 and 7 mA.

For some applications, the device includes an environmentally-sensitive coating that dissolves when the device reaches a certain area of the GI tract, and the signal controller is configured to detect that the coating has dissolved, and to drive the electrodes responsively to the detection. For some applications, the device includes an optical sensor which is configured to detect light projected from outside a body of the subject, and the signal controller is configured to begin driving the electrodes responsively to the detection.

For some applications, the signal controller is configured to drive the electrodes to apply a voltage drop between two of the electrodes to be between 0.4 and 8.4 volts. Alternatively or additionally, the signal controller is configured to drive the electrodes to apply a voltage drop between two of the electrodes that is between 1 and 3 volts. Further alternatively or additionally, the signal controller is configured to drive the electrodes to apply the signal with a characteristic frequency of between 7 and 30 Hz, such as between 10 and 30 Hz, e.g., between 10 and 20 Hz.

In an embodiment, the device includes a sensor, configured to detect a property of the GI tract in a vicinity of the device, and to generate a sensor signal responsively to the property, and the signal controller is configured to begin driving the electrodes responsively to the sensor signal. For some applications, the property includes inflammation of the GI tract, and the sensor is configured to detect the inflammation, and to generate the sensor signal responsively thereto. For example, the sensor may include an optical sensor, configured to detect the inflammation.

In an embodiment, the signal controller is configured to receive an indication regarding a disposition of the device within the GI tract, and to begin driving the electrodes responsively to the indication. For some applications, the device includes a timer, which is configured to generate the indication responsively to a duration of the device in the GI tract.

There is further provided, in accordance with an embodiment of the present invention, a method including:

identifying that a subject may benefit from increased local endogenous release of NO;

orally administering an ingestible device to the subject;

applying, from the device, an electrical signal to an inner surface of a wall of a gastrointestinal (GI) tract of a subject; and

configuring the signal to induce local endogenous release of nitric oxide (NO) in the GI tract.

For some applications, the method includes projecting light from outside a body of the subject towards a certain area of the GI tract; and detecting, at the device, the projected light, and applying the signal includes beginning to apply the signal responsively to the detection.

In an embodiment, identifying includes identifying that the subject may benefit from the increased local endogenous release of the NO to a site in the GI tract.

For some applications, identifying includes identifying that the subject may benefit from at least one of: improved gastrointestinal mucosal integrity, and a reduced likelihood of acute microvascular injuries. For some applications, identifying includes identifying that the subject may benefit from at least one of: modulated mucus secretion, and modulated alkaline secretion. For some applications, identifying includes identifying that the subject may benefit from improved blood flow in at least one of: gastric mucosa, a mesenteric vascular bed, and an area of intestinal tissue. For some applications, identifying includes identifying that the subject may benefit from increased vasodilation of surrounding GI vasculature. For some applications, identifying includes identifying that the subject may benefit from at least one of: an attenuated inflammatory response, and improved microvascular reactions occurring in the GI tract wall. For some applications, identifying includes identifying that the subject suffers from a condition selected from the group consisting of: GI inflammation, sepsis, irritable bowel syndrome (IBS), Crohn's disease, and an inflammatory disorder.

For some applications, identifying includes identifying that the subject may benefit from down-regulation of an immune response during a condition selected from the group consisting of: an inflammatory condition, and an immunogenic condition.

For some applications, identifying includes identifying that the subject may benefit from regulation of muscle tone of at least one of: a GI sphincter of the subject, a peristaltic reflex of a stomach of the subject, and a peristaltic reflex of an intestine of the subject. For some applications, identifying includes identifying that the subject suffers from a motility disorder of the GI tract.

In an embodiment, identifying includes identifying that the subject may benefit from a systemic effect caused by the local release of the NO. For some applications, identifying includes identifying that the subject may benefit from a systemic anti-inflammatory effect caused by the local release of the NO. For some applications, identifying includes identifying that the subject suffers from an inflammatory disease. For some applications, identifying includes identifying that the subject may benefit from improved endothelial function.

For some applications, identifying includes identifying that the subject suffers from a condition selected from the group consisting of: hypertension, atherosclerosis, hypercholesterolemia, a peripheral vascular disease, coronary artery disease, and a urogenital disorder. For some applications, the effect is selected from the group consisting of: an inhibitory effect on platelet aggregation, and an anticoagulatory effect, and identifying includes identifying that the subject may benefit from the selected effect. For some applications, identifying includes identifying that the subject suffers from a coagulation-anticoagulation imbalance. For some applications, the effect includes a systemic antioxidative effect, and identifying includes identifying that the subject may benefit from the systemic antioxidative effect.

For some applications, identifying includes identifying that the subject suffers from diabetes. For example, the effect may include an effect on insulin sensitivity, and identifying may include identifying that the subject may benefit from the effect on insulin sensitivity.

The present invention will be more fully understood from the following detailed description of embodiments thereof, taken together with the drawings, in which:

### BRIEF DESCRIPTION OF THE DRAWINGS

FIG. 1 is a schematic illustration of an ingestible electrical-stimulation device, in accordance with an embodiment of the present invention; and

FIGS. 2-5 are graphs showing in vitro experimental results measured in accordance with respective embodiments of the present invention.

### DETAILED DESCRIPTION OF EMBODIMENTS

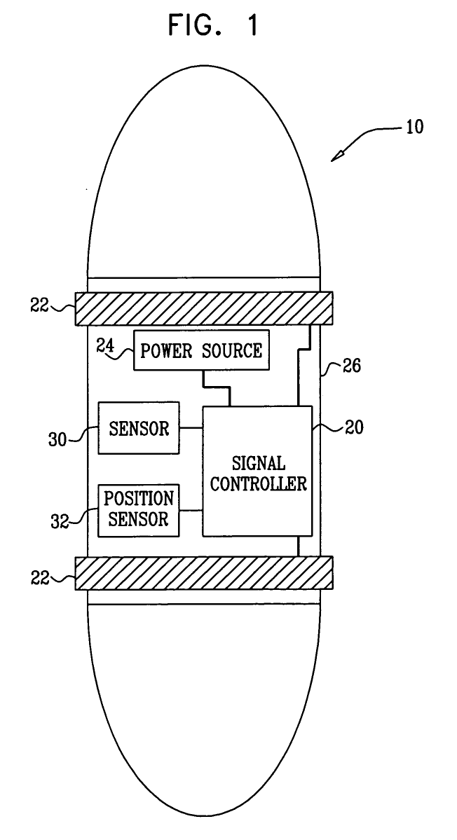

FIG. 1 is a schematic illustration of an ingestible electrical-stimulation device 10, in accordance with an embodiment of the present invention. Device 10 comprises a signal controller 20, one or more electrodes 22, a power source 24, and a housing 26. Housing 26 comprises a biocompatible, biologically inert material, such as stainless steel or silicone, which is typically shaped so as to define a smooth outer surface, so as to avoid damage to gastrointestinal (GI) tissue as the device travels through the GI tract. For example, housing 26 may be shaped similarly to a conventional drug capsule. After ingestion, device 10 typically is propelled through the GI tract by the normal peristaltic motion of the GI tract. Alternatively, the device regulates its rate of transport through the GI tract by modulating local peristaltic waves, such as using techniques described in one or more of the references mentioned hereinabove in the Background of the Invention.

Signal controller 20 is configured to apply an electrical signal intraluminally to an inner surface of a wall of the GI tract. Signal controller 20 configures the signal to induce local endogenous release of nitric oxide (NO) in the GI tract, in order to treat a local or a systemic condition. Typically, the signal is configured to stimulate mucosal and submucosal neuronal complexes, thereby activating neuronal NO synthase (NNOS) and/or submucosal endothelial NO synthase (eNOS). “Induce,” as used in the present application, including in the claims, is to be understood as including both inducing NO production, and enhancing NO production that would have occurred even in the absence of the application of the techniques described herein.

For some applications, signal controller 20 applies the signal as a pulsed DC train, which is monophasic or biphasic, and has relatively low duty cycle values and low amplitudes. For example, the signal may include a monophasic DC pulse train of pulses, each of which has a duration of between about 0.1 and 1 ms, e.g., about 1 ms, at a frequency of between about 7 and about 50 Hz, e.g., about 18 Hz, and having regulated current of between about 2 and about 7 mA, e.g., about 5 mA. For some applications, signal controller 20 comprises circuitry configured to regulate electrical signal delivery to a desired current level, rather than a desired voltage level.

The electrically-induced local release of NO in the GI tract generally:
maintains gastrointestinal mucosal integrity and prevents acute microvascular injuries induced by endotoxins, ischemic factors, and various irritants;
modulates mucus and alkaline secretion, thereby enhancing the GI tract's viscoelastic protective layer and accelerating healing of preexisting ulcers in the GI wall;
improves blood flow in the gastric mucosa, the mesenteric vascular bed, and various areas of the intestinal tissue, thereby contributing to the maintenance of mucosal integrity;
causes vasodilation of the surrounding GI vasculature, thereby causing increased perfusion of tissue, which has local anti-necrotic and anti-inflammatory effects;
attenuates inflammatory response and improves microvascular reactions occurring in the GI wall during various pathological conditions, such as GI inflammation, sepsis, irritable bowel syndrome (IBS), Crohn's disease, and other inflammatory disorders;
down-regulates the immune response during various inflammatory and immunogenic conditions;
regulates muscle tone of the GI sphincters, and the peristaltic reflex of the stomach and the intestine, including gastric emptying and intestinal transit, thereby treating various motility disorders of the gastrointestinal tract; and/or
has beneficial effects outside of the GI tract, such as:
   systemic anti-inflammatory effects beneficial for treating inflammatory diseases;
   improvement of endothelial function with consequent vasodilatation and increases in blood flow, in various disease states such as hypertension, atherosclerosis, hypercholesterolemia, diabetes, peripheral vascular diseases, coronary artery diseases, and urogenital disorders;
   an inhibitory effect on platelet aggregation and an anticoagulatory effect, which are beneficial for treating a coagulation-anticoagulation imbalance in various pathological states;
   a systemic antioxidative effect by reducing free radical reactions and stimulating antioxidative enzymes, which provides therapeutic benefits for various disorders associated with increased formation of free radicals, such as atherosclerosis, peripheral vascular disorders, and diabetes; and/or
   a beneficial effect on insulin sensitivity in diabetes.

For some applications, power source 24 comprises one or more batteries, such as silver oxide batteries or other batteries that do not require oxygen to operate. For other applications, power source 24 comprises a transducer configured to receive power wirelessly transmitted from a transmitter positioned outside of the subject's body, such as by using induction, RF energy, or ultrasound energy.

In an embodiment of the present invention, signal controller 20 is configured to receive an indication of a parameter of (a) the GI tract in a vicinity of device 10, and/or (b) a location of device 10 within the GI tract, and to apply the electrical signal responsively to the indication. For some applications, the indication indicates that the device has reached the small intestine or the large intestine.

For some applications, device 10 comprises a sensor 30, which is configured to sense a parameter of the GI tract in the vicinity of the device. Signal controller 20 is configured to begin and/or end application of the electrical signal responsively to the sensed physiological parameter. For some applications, sensor 30 comprises:
an enzymatic sensor, which is selectively sensitive to an enzyme indicative of the device's presence in a given portion of the GI tract and/or sensitive to a pathological condition, such as inflammation or GI bleeding;
a temperature sensor, e.g., a sensor sensitive to elevated temperatures associated with inflammation;
a pH sensor, e.g., a pH sensor sensitive to a particular pH in the range of about 4.7-6.5;
a pressure sensor;
an optical sensor; or
a chemical sensor, which senses a concentration of a chemical in the GI tract, such as glucose or a particular drug.

In an embodiment of the present invention, sensor 30 comprises an optical sensor configured to detect light projected from outside of the body of the subject, and signal controller 20 applies the signal responsively to the detection. For some applications, a healthcare worker applies a light source to an external surface of the subject's body in a vicinity of a portion of the GI tract at which signal controller 20 is to apply the signal. For example, the healthcare worker may apply the light source to an external surface in a vicinity of an inflamed portion of the GI tract.

Alternatively or additionally, device 10 comprises an environmentally-sensitive coating (e.g., a pH-sensitive coating) that dissolves when the device reaches a certain area of the GI tract, such as the duodenum. Signal controller 20 is configured to detect that the coating has dissolved, and apply the signal responsively to the detection.

For some applications, device 10 comprises a position sensor 32, which is adapted to sense a position of the device within the GI tract. Signal controller 20 is configured to begin and/or end application of the electrical signal responsively to the sensed position.

For some applications, signal controller 20 comprises a timer, and the signal controller is configured to begin and/or end application of the stimulation responsively to a value of the timer. For some applications, signal controller 20 begins application of the stimulation responsively to one or more of the indications described above, and applies the stimulation for a period times by the timer.

In an embodiment of the present invention, device 10 is configured to contain a drug for delivery to the GI tract. The device is typically configured to release the drug generally at the same time that signal controller 20 applies the NO-release-inducing signal to the GI tract. Typically, but not necessarily, the signal applied by signal controller 20 does not enhance absorption of the drug. For some applications, the drug includes an anti-inflammatory drug.

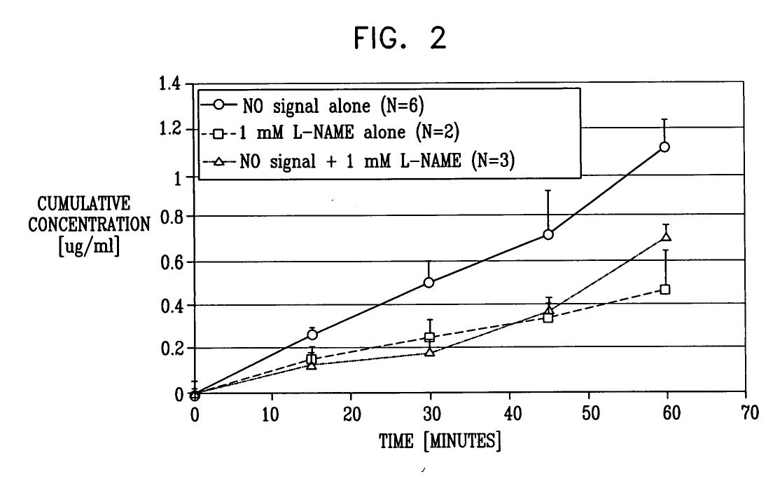

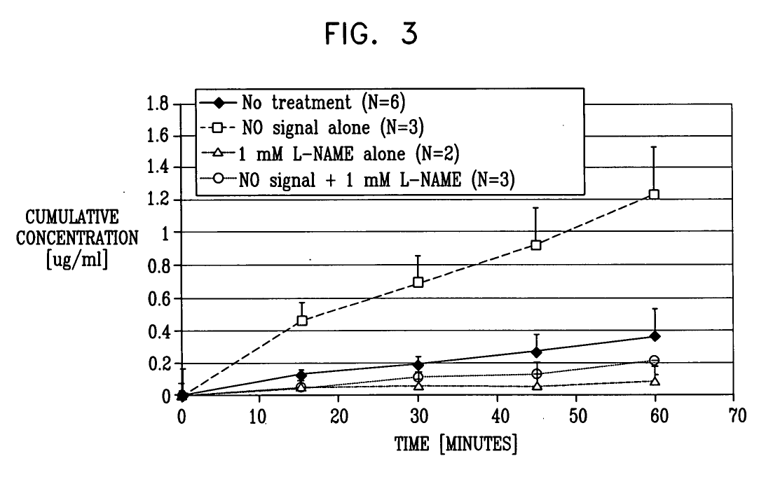

Reference is made to FIGS. 2 and 3, which are graphs showing in vitro experimental results measured in accordance with an embodiment of the present invention. These experiments assessed the effect of the application of an electrical signal configured to enhance NO-associated drug permeation, which is inhibited by non-specific NOS inhibitor NG-Nitro-L-Arginine methyl ester (L-NAME) in rat jejunum in vitro. The NO-releasing electrical signal (hereinbelow, the “NO signal”) was applied with the following parameters: an amplitude of 5 mA, a pulse width of 1 ms, and a frequency of 18 Hz.

In the experiment shown in FIG. 2, 1 mg/ml octreotide was applied to 11 segments of rat jejunum. The NO signal alone was applied to six of the segments, 1 mM L-NAME alone was applied to two of the segments, and the NO signal and 1 mM L-NAME were applied to three of the segments. In the experiment shown in FIG. 3, 1 mg/ml leuprolide was applied to 14 segments of rat jejunum. The NO signal alone was applied to three of the segments, 1 mM L-NAME alone was applied to two of the segments, the NO signal and 1 mM L-NAME were applied to three of the segments, and no treatment was applied to the remaining six segments.

As can be seen in the figures, the application of the NO signal substantially enhanced drug permeation, while L-NAME nearly prevented this enhanced drug permeation. These results indicate that the NO signal described herein induces the release of NO, and that the electrically-stimulated release of NO plays an important role in electrically-stimulated drug absorption in intestinal tissue.

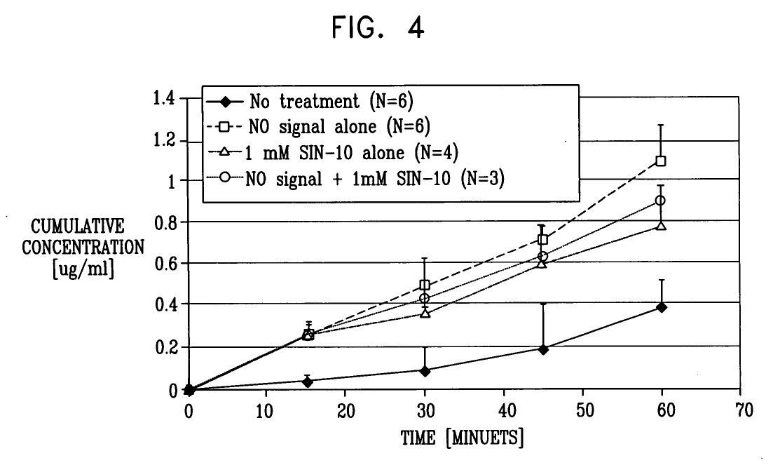

FIG. 4 is a graph showing in vitro experimental results measured in accordance with an embodiment of the present invention. In this experiment, the permeation-enhancing effect of electrical stimulation in rat jejunum in vitro was compared with the effect of an NO donor, molsidomine (SIN-10) (exogenous nitric oxide). The NO signal alone was applied to six segments of rat jejunum, 1 mM SIN-10 alone was applied to four segments, the NO signal and 1 mM SIN-10 were applied to three segments, and no treatment was applied to six segments. As can be seen in the figure, the rate of octreotide transepithelial transport in the presence of SIN-10 was similar to the electrically-induced absorption of the same peptide. The combination of electrical stimulation with SIN-10 incubation did not augment the enhanced permeation of octreotide achieved with electrical stimulation alone. These results indicate that electrical stimulation may induce endogenous release of NO that facilitates transepithelial absorption similarly to that achieved by exogenous NO released from an NO donor.

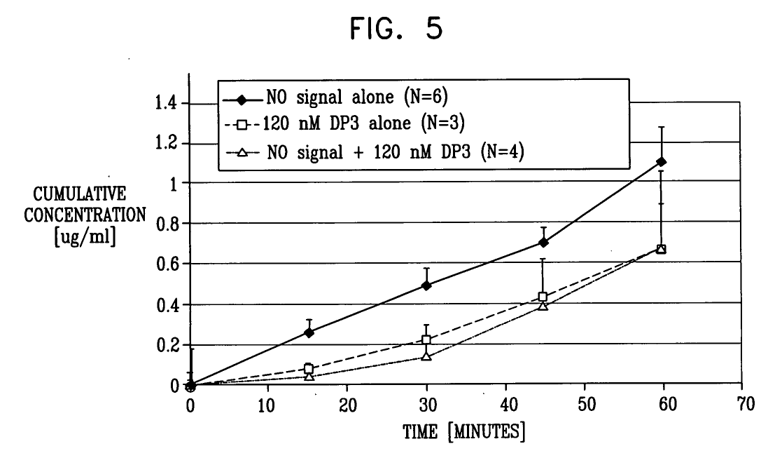

FIG. 5 is a graph showing in vitro experimental results measured in accordance with an embodiment of the present invention. In this experiment, the role of neuronal NO synthase (NNOS) in electrically-stimulated octreotide absorption was investigated by using a potent nNOS-selective inhibitor—(4S)-N-(4-amino-5-[aminoethyl]aminopentyl)-N′-nitroguanidine (DP3) (Hah et al., 2001). The NO signal alone was applied to six segments of rat jejunum, 120 nM DP3 alone was applied to three segments, and the NO signal and 120 nM DP3 were applied to four segments. As can be seen in the figure, the addition of DP3 substantially inhibited the electrically-mediated transepithelial transport of octreotide. These results indicate that nNOS plays a role in mediating electrical stimulation applied to the intestinal mucosal layer.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
International Patent Application PCT/IL2004/000093, filed Jan. 29, 2004;
U.S. patent application Ser. No. 10/767,663, filed Jan. 29, 2004;
U.S. Provisional Patent Application 60/668,738, filed Apr. 5, 2005;
U.S. patent application Ser. No. 10/838,072, filed May 3, 2004;
U.S. patent application Ser. No. 10/901,742, filed Jul. 29, 2004;
U.S. Provisional Patent Application 60/636,447, filed Dec. 14, 2004;
International Patent Application PCT/IL2005/000301, filed Mar. 16, 2005;
International Patent Application PCT/IL05/01346, filed Dec. 14, 2005; and/or
International Patent Application PCT/IL05/01347, filed Dec. 14, 2005.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising an ingestible device, which comprises:
two or more electrodes; and
a signal controller, configured to drive the electrodes to apply an electrical signal to an inner surface of a wall of a gastrointestinal (GI) tract of a subject, and to configure the signal to induce local endogenous release of nitric oxide (NO) in the GI tract.

2. The apparatus according to claim 1, wherein the signal controller is configured to configure the signal to stimulate neuronal complexes of the GI tract selected from the group consisting of: mucosal neuronal complexes, and submucosal neuronal complexes.

3. The apparatus according to claim 1, wherein the signal controller is configured to drive the electrodes to apply the signal with an amplitude of between 2 and 7 mA.

4. The apparatus according to claim 1, wherein the device comprises an environmentally-sensitive coating that dissolves when the device reaches a certain area of the GI tract, and wherein the signal controller is configured to detect that the coating has dissolved, and to drive the electrodes responsively to the detection.

5. The apparatus according to claim 1, wherein the device comprises an optical sensor which is configured to detect light projected from outside a body of the subject, and wherein the signal controller is configured to begin driving the electrodes responsively to the detection.

6. The apparatus according to claim 1, wherein the signal controller is configured to drive the electrodes to apply a voltage drop between two of the electrodes to be between 0.4 and 8.4 volts.

7. The apparatus according to claim 6, wherein the signal controller is configured to drive the electrodes to apply a voltage drop between two of the electrodes that is between 1 and 3 volts.

8. The apparatus according to claim 1, wherein the signal controller is configured to drive the electrodes to apply the signal with a characteristic frequency of between 7 and 30 Hz.

9. The apparatus according to claim 8, wherein the signal controller is configured to drive the electrodes to apply the signal with a characteristic frequency of between 10 and 30 Hz.

10. The apparatus according to claim 9, wherein the signal controller is configured to drive the electrodes to apply the signal with a characteristic frequency of between 10 and 20 Hz.

11. The apparatus according to claim 1, wherein the device comprises a sensor, configured to detect a property of the GI tract in a vicinity of the device, and to generate a sensor signal responsively to the property, and wherein the signal controller is configured to begin driving the electrodes responsively to the sensor signal.

12. The apparatus according to claim 11, wherein the property includes inflammation of the GI tract, and wherein the sensor is configured to detect the inflammation, and to generate the sensor signal responsively thereto.

13. The apparatus according to claim 12, wherein the sensor comprises an optical sensor, configured to detect the inflammation.

14. The apparatus according to claim 1, wherein the signal controller is configured to receive an indication regarding a disposition of the device within the GI tract, and to begin driving the electrodes responsively to the indication.

15. The apparatus according to claim 14, wherein the device comprises a timer, which is configured to generate the indication responsively to a duration of the device in the GI tract.

16. A method comprising:
identifying that a subject may benefit from increased local endogenous release of NO;
orally administering an ingestible device to the subject;
applying, from the device, an electrical signal to an inner surface of a wall of a gastrointestinal (GI) tract of a subject; and
configuring the signal to induce local endogenous release of nitric oxide (NO) in the GI tract.

17. The method according to claim 16, wherein configuring the signal comprises configuring the signal to stimulate neuronal complexes of the GI tract selected from the group consisting of: mucosal neuronal complexes, and submucosal neuronal complexes.

18. The method according to claim 16, wherein applying the signal comprises configuring the signal to have an amplitude of between 2 and 7 mA.

19. The method according to claim 16, wherein the device includes an environmentally-sensitive coating that dissolves when the device reaches a certain area of the GI tract, and wherein applying the signal comprises detecting that the coating has dissolved, and applying the signal responsively to the detection.

20. The method according to claim 16, comprising:
projecting light from outside a body of the subject towards a certain area of the GI tract; and
detecting, at the device, the projected light,
wherein applying the signal comprises beginning to apply the signal responsively to the detection.

21. The method according to claim 16, wherein applying the signal comprises applying a voltage drop of between 0.4 and 8.4 volts.

22. The method according to claim 21, wherein applying the signal comprises applying a voltage drop of between 1 and 3 volts.

23. The method according to claim 16, wherein applying the signal comprises configuring the signal to have a characteristic frequency of between 7 and 30 Hz.

24. The method according to claim 23, wherein applying the signal comprises configuring the signal to have a characteristic frequency of between 10 and 30 Hz.

25. The method according to claim 24, wherein applying the signal comprises configuring the signal to have a characteristic frequency of between 10 and 20 Hz.

26. The method according to claim 16, wherein applying the signal comprises detecting a property of the GI tract in a vicinity of the device, and beginning to apply the signal responsively to the detecting.

27. The method according to claim 26, wherein the property includes inflammation of the GI tract, and wherein detecting comprises detecting the inflammation.

28. The method according to claim 27, wherein detecting comprises optically detecting the inflammation.

29. The method according to claim 16, wherein applying the signal comprises receiving an indication regarding a disposition of the device within the GI tract, and beginning to apply the current responsively to the indication.

30. The method according to claim 29, wherein applying the signal comprises applying the signal responsively to a duration of the device in the GI tract.

31. The method according to claim 16, wherein identifying comprises identifying that the subject may benefit from the increased local endogenous release of the NO to a site in the GI tract.

32. The method according to claim 16, wherein identifying comprises identifying that the subject may benefit from at least one of: improved gastrointestinal mucosal integrity, and a reduced likelihood of acute microvascular injuries.

33. The method according to claim 16, wherein identifying comprises identifying that the subject may benefit from at least one of: modulated mucus secretion, and modulated alkaline secretion.

34. The method according to claim 16, wherein identifying comprises identifying that the subject may benefit from improved blood flow in at least one of: gastric mucosa, a mesenteric vascular bed, and an area of intestinal tissue.

35. The method according to claim 16, wherein identifying comprises identifying that the subject may benefit from increased vasodilation of surrounding GI vasculature.

36. The method according to claim 16, wherein identifying comprises identifying that the subject may benefit from at least one of: an attenuated inflammatory response, and improved microvascular reactions occurring in the GI tract wall.

37. The method according to claim 16, wherein identifying comprises identifying that the subject suffers from a condition selected from the group consisting of: GI inflammation, sepsis, irritable bowel syndrome (IBS), Crohn's disease, and an inflammatory disorder.

38. The method according to claim 16, wherein identifying comprises identifying that the subject may benefit from down-regulation of an immune response during a condition selected from the group consisting of: an inflammatory condition, and an immunogenic condition.

39. The method according to claim 16, wherein identifying comprises identifying that the subject may benefit from regulation of muscle tone of at least one of: a GI sphincter of the subject, a peristaltic reflex of a stomach of the subject, and a peristaltic reflex of an intestine of the subject.

40. The method according to claim 16, wherein identifying comprises identifying that the subject suffers from a motility disorder of the GI tract.

41. The method according to claim 16, wherein identifying comprises identifying that the subject may benefit from a systemic effect caused by the local release of the NO.

42. The method according to claim 41, wherein identifying comprises identifying that the subject may benefit from a systemic anti-inflammatory effect caused by the local release of the NO.

43. The method according to claim 41, wherein identifying comprises identifying that the subject suffers from an inflammatory disease.

44. The method according to claim 41, wherein identifying comprises identifying that the subject may benefit from improved endothelial function.

45. The method according to claim 41, wherein identifying comprises identifying that the subject suffers from a condition selected from the group consisting of: hypertension, atherosclerosis, hypercholesterolemia, a peripheral vascular disease, coronary artery disease, and a urogenital disorder.

46. The method according to claim 41, wherein the effect is selected from the group consisting of: an inhibitory effect on platelet aggregation, and an anticoagulatory effect, and wherein identifying comprises identifying that the subject may benefit from the selected effect.

47. The method according to claim 41, wherein identifying comprises identifying that the subject suffers from a coagulation-anticoagulation imbalance.

48. The method according to claim 41, wherein the effect includes a systemic antioxidative effect, and wherein identifying comprises identifying that the subject may benefit from the systemic antioxidative effect.

49. The method according to claim 41, wherein identifying comprises identifying that the subject suffers from diabetes.

50. The method according to claim 49, wherein the effect includes an effect on insulin sensitivity, and wherein identifying comprises identifying that the subject may benefit from the effect on insulin sensitivity.
